(19) United States
(12) Patent Application Publication
(10) Pub. No.: US 2007/0244494 A1
(43) Pub. Date: Oct. 18, 2007

(54) Methods and devices for treating atrial septal defects

(76) Inventor: Stephen W. Downing, Buffalo, NY (US)

Correspondence Address: MORRISON & FOERSTER LLP, 755 PAGE MILL RD, PALO ALTO, CA, 94304-1018, US

(21) Appl. No.: 11/407,326

(22) Filed: Apr. 18, 2006

(51) Int. Cl. A61B 17/08

(52) U.S. Cl. 606/151

(57) ABSTRACT

Disclosed herein are devices and methods for treating a defect in the septum between the right and left atria of a subject's heart. In one approach, an implantable device is positioned in the right atrium with a first end in the superior vena cava, a second end in the inferior vena cava, and at least a part of a central portion at least partially covering a defect. In another approach, a patch is positioned in the right atrium to cover substantially all of the right atrium side of the interatrial septum without blocking a valve or coronary sinus.

### FIELD

Described herein are methods and devices for treating defects in the septum between the right and left atria of a subject's heart. Such defects include, but are not limited to, atrial septal defects, septal aneurysms, and patent foramen ovale. Atrial septal defects include, but are not limited to, secundum defects, upper sinus venosus defects, and lower sinus venosus defects. These methods and apparatus may also be used to treat a patent ductus arteriosus.

### BACKGROUND

The interatrial septum is a thin wall of tissue that separates the right and left atria of the heart. A number of defects can occur in the interatrial septum including, for example, holes and aneurysms. In some cases, the presence of such atrial septal defects can allow shunting of blood between the right and left atria and consequently require the heart to work harder. In addition, some atrial septal defects may allow blood clots, embolic matter, or other debris to pass from the right atrium to the left atrium. Clots or embolic material formed on or in a defect in the interatrial septum may also pass into the left atrium. Such material passing into the left atrium bypasses the lungs, which might otherwise have captured it, and can subsequently reach the brain and cause a stroke.

A patent foramen ovale (PFO) is a frequently occurring defect in the interatrial septum. The foramen ovale is an opening in the interatrial septum that allows blood to pass between the atria of the fetal heart. Typically, after birth the fetal septum primum and septum secundum fuse to close the foramen ovale and form the interatrial septum. A patent foramen ovale results when the septum primum and septum secundum do not completely fuse, but instead form a flap valve that can in some cases open when the pressure in the right atrium exceeds that in the left atrium.

Patent foramen ovale defects are associated with cryptogenic strokes (i.e., strokes of unknown cause) and migraine headaches. In particular, approximately 40% of the 150,000 ischemic strokes that occur each year in the U.S. are cryptogenic, and approximately 50% of patients with cryptogenic stroke have a patent foramen ovale. Although the mechanisms linking patent foramen ovale to cryptogenic stroke and migraine headache are debated, it has been demonstrated that treating patent foramen ovale defects reduces stroke incidence and migraine symptoms and occurrence.

### SUMMARY

Disclosed herein are devices and methods for treating a defect in the septum between the right and left atria of a subject's heart. A defect may be treated, for example, by at least partially covering, mending, closing, blocking, and/or stabilizing it with an implantable device. In some variations a septal aneurysm may be stabilized with an implantable device. The stabilized septal aneurysm may occur in combination with another interatrial septal defect such as a patent foramen ovale, for example, that is treated with the same device.

In one approach, a method for treating a defect in the septum between the right and left atria of a subject's heart comprises providing an implantable device including a first end, a second end, and a central portion, and positioning the device in the right atrium with the first end in the subject's superior vena cava, the second end in the subject's inferior vena cava, and at least a part of the central portion at least partially covering the defect. In some variations the defect is entirely covered with at least a part of the central portion of the device.

As used herein, the term “cover” means to place over or upon in either a partial or complete fashion. A part of the central portion of the device may partially or entirely cover the defect without the central portion of the device making contact with the defect or surrounding regions of the interatrial septum. However, in some variations partially or entirely covering the defect includes contacting the defect and/or surrounding regions of the interatrial septum with a part of the central portion of the device.

In partially or entirely covering the defect, a part of the central portion of the device may partially or entirely seal the defect and thus inhibit or prevent the flow of blood and other material from or through the defect into the right atrium. In some variations, however, some or all of the central portion of the device is permeable to blood flow. In some variations, the part of the central portion of the device covering the defect is permeable to blood flow but inhibits or prevents the passage of other material, such as clots and embolic material, for example, from or through the defect into the right atrium.

The step of covering the defect may include covering the entire fossa ovalis with at least a part of the central portion of the device without blocking a valve or coronary sinus. In some variations, covering the defect includes covering substantially all of the right atrium side of the interatrial septum with at least part of the central portion of the device without blocking a valve or coronary sinus. As used herein, “substantially all of the right atrium side of the interatrial septum” may comprise, for example, greater than about 50%, 60%, 70%, 80%, or 90% of the right atrium side of the interatrial septum excluding valves and coronary sinus. In some variations, 100% of the interatrial septum is covered excluding valves and coronary sinus.

The method may further comprise securing at least a part of the central portion of the device to the interatrial septum at one or more locations proximate the defect. This may be accomplished, for example, by placing at least one anchor through at least a part of the central portion of the device into the interatrial septum. In other variations, at least a part of the central portion of the device may be secured to the interatrial septum with adhesives or sutures, for example.

In some variations, the implantable device may be introduced into the right atrium during open heart surgery. In other variations, the device may be introduced intravascularly. Where the device is introduced intravascularly, the method may further comprise compressing the implantable device to fit into an intravascular delivery device, introducing the implantable device into the right atrium using the delivery device, expanding the first end of the implantable device to securely position the first end in the superior vena cava, expanding the central portion of the implantable device to position at least a part of the central portion to at least partially cover the defect, and expanding the second end of the implantable device to securely position the second end in the inferior vena cava.

In variations in which the implantable device is introduced into the right atrium intravascularly, the method may further comprise intravascularly securing at least a part of the central portion of the implantable device to the septum at one or more locations proximate the defect. This may be accomplished, for example, by delivering the implantable device with a first intravascular delivery device and delivering at least one anchor with the same intravascular delivery device or with a second intravascular delivery device. The anchor may be placed through at least a part of the central portion of the implantable device into the septum.

A device for treating a defect in the septum between the right and left atria of a subject's heart comprises, in some variations, a first end expandable to be securely positioned in the subject's inferior vena cava, a second end expandable to be securely positioned in the subject's superior vena cava, and a central portion expandable to position at least a part of the central portion to at least partially cover the defect when the first end is securely positioned in the subject's inferior vena cava and the second end is securely positioned in the subject's superior vena cava. In some variations, the device may be compressed to fit in an intravascular delivery device with which it is introduced into the right atrium.

The device may comprise one or more materials that promote tissue ingrowth into the device and/or are conformable to irregularities in atrial surfaces to promote formation of a seal against those surfaces. The device may also comprise a framework formed, for example, from conventional shape memory materials. The framework may be partially or entirely covered or coated with materials that are conformable to atrial surfaces and/or promote tissue ingrowth. Some or all of the materials from which the device is constructed may be bioabsorbable.

In some variations, at least a part of the central portion of the device has a concave shape positionable to face and at least partially cover the defect. A septal side of the concave portion may comprise materials that are conformable to atrial surfaces and/or promote tissue ingrowth.

In another approach, a method for treating a defect in the septum between the right and left atria of a subject's heart comprises providing a patch, and covering substantially all of the right atrium side of the septum with the patch without blocking a valve or coronary sinus. As used herein, the term “patch” means an implantable device that may be used to at least partially cover, mend, close, block, and/or stabilize a defect in the septum between the right and left atria.

In some variations, the patch is positioned so that a concave portion of the patch faces and at least partially covers the defect. The patch may be introduced into the right atrium intravascularly, for example. In one variation, the method comprises delivering the patch to the right atrium with a first intravascular delivery device, and delivering at least one anchor to anchor the patch to the septum with a second intravascular delivery device.

In some variations, a septal side of the patch comprises one or more materials that are conformable to irregularities in atrial surfaces and/or promote tissue ingrowth. In some variations, some or all of the materials from which the patch is constructed may be bioabsorbable.

In one variation, an implantable device as described above includes a patch as just described as part of the central portion of the device. A method for treating a defect in the interatrial septum may then comprise, for example, positioning the central portion of the implantable device in the right atrium such that the patch covers substantially all of the right atrium side of the septum without blocking a valve or coronary sinus.

In some variations, a septal aneurysm may be stabilized by introducing an implantable device and/or patch as described above into the right atrium, and then securing at least part of the septal aneurysm to at least a portion of the implantable device and/or patch.

Once secured in position in the right atrium, an implantable device as described above (optionally including a patch as described above) may advantageously direct blood flow away from the septal defect.

Treating a defect in the interatrial septum by covering substantially all of the right atrium side of the interatrial septum with a patch or other implantable device may, in some variations, reduce the precision with which the patch or device must be placed to effect treatment, and may make the treatment less sensitive to variations in individual anatomy. Imaging resolution requirements may consequently be relaxed, as well. Use of a patch or other implantable device comprising conformable (e.g., soft), bioabsorbable, and/or tissue ingrowth promoting material may, in some variations, reduce the risk that the device will erode into surrounding tissue.

In some variations, implantable devices and/or patches similar to or identical to those described above may be used to treat a patent ductus arteriosus (failure of a connection between the aorta and the pulmonary artery to close at birth).

These and other embodiments, features and advantages will become more apparent to those skilled in the art when taken with reference to the following more detailed description in conjunction with the accompanying drawings that are first briefly described.

### BRIEF DESCRIPTION OF THE DRAWINGS

FIGS. 1A and 1B show one example of an implantable device that may be used to treat a defect in a subject's interatrial septum in compressed and expanded configurations, respectively.

FIGS. 2A and 2B show, respectively, top and side views of one variation of an implantable device for treating defects in the interatrial septum being positioned with an intravascular delivery device in the right atrium of a subject's heart with one end in the inferior vena cava and another end in the superior vena cava.

FIGS. 3A and 3B show, respectively, top and side views of the implantable device of FIGS. 2A and 2B expanding as it is deployed by the delivery device.

FIGS. 4A and 4B show, respectively, top and side views of the implantable device of FIGS. 2A and 2B covering a patent foramen ovale after deployment into the right atrium, inferior vena cava, and superior vena cava.

FIGS. 5A and 5B show, respectively, top and side views of an anchor being placed through a portion of the implantable device of FIGS. 2A and 2B into the interatrial septum to secure the device to the septum.

FIGS. 6A and 6B show, respectively, top and side views of the implantable device of FIGS. 2A and 2B secured to the interatrial septum with anchors.

FIGS. 7A-7C show additional examples of expanded configurations for implantable devices that may be used to treat a defect in a subject's interatrial septum.

FIGS. 8A and 8B show, respectively, side and perspective views of the expanded configuration of a variation of an implantable device including a patch that may be used to treat a defect in a subject's interatrial septum.

FIGS. 9A and 9B show, respectively, top and side views of a variation of the device of FIGS. 8A and 8B covering a patent foramen ovale after deployment in the right atrium, inferior vena cava, and superior vena cava.

FIG. 10 shows a cross-sectional perspective view of one variation of an implantable patch that may be used to treat a defect in a subject's interatrial septum.

FIGS. 11A and 11B show, respectively, top and side views of one variation of an implantable patch for treating defects in the interatrial septum being positioned with an intravascular delivery device in the right atrium of a subject's heart.

FIGS. 12A and 12B show, respectively, top and side views of the patch of FIGS. 11A and 11B deployed and expanded in the right atrium to cover a patent foramen ovale.

FIGS. 13A and 13B show, respectively, top and side views of an anchor being placed through the patch of FIGS. 11A and 11B into the interatrial septum to secure the patch to the septum.

FIGS. 14A and 14B show, respectively, top and side views of the patch of FIGS. 11A and 11B secured to the interatrial septum with anchors.

### DETAILED DESCRIPTION

The following detailed description should be read with reference to the drawings, in which identical reference numbers refer to like elements throughout the different figures. The drawings, which are not necessarily to scale, depict selective embodiments and are not intended to limit the scope of the invention. The detailed description illustrates by way of example, not by way of limitation, the principles of the invention. As used in the specification and the appended claims, the singular forms “a,” “an” and “the” include plural referents unless the context clearly indicates otherwise. This description will clearly enable one skilled in the art to make and use the invention, and describes several embodiments, adaptations, variations, alternatives and uses of the invention, including what is presently believed to be the best mode of carrying out the invention.

Disclosed herein are examples and variations of methods and devices that may be used to treat defects in the interatrial septum of a subject's heart. For convenience of illustration, these methods and devices will be described primarily with respect to treatment of a patent foramen ovale. One of ordinary skill in the art having the benefit of this disclosure will appreciate that these methods and devices can be used or adapted to be used to treat other defects in the interatrial septum including, but not limited to, atrial septal defects and septal aneurysms. Atrial septal defects include, but are not limited to, secundum defects, upper sinus venosus defects, and lower sinus venosus defects.

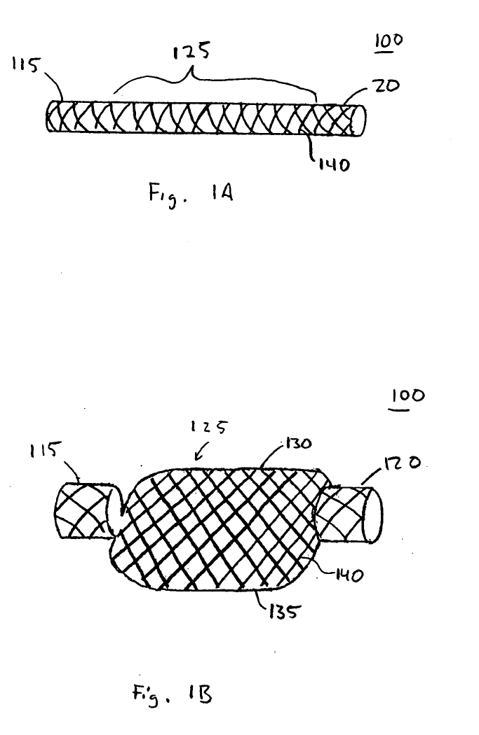

One example of an implantable device 100 that may be used to treat a defect in the interatrial septum is shown in FIGS. 1A and 1B in, respectively, compressed and expanded configurations. In this example, device 100 comprises a first end 115 that may be expanded to be securely positioned in a subject's inferior vena cava, a second end 120 that may be expanded to be securely positioned in the subject's superior vena cava, and a central portion 125 that may be expanded to at least partially cover the defect in the interatrial septum when the first end is securely positioned in the inferior vena cava and the second end is securely positioned in the subject's superior vena cava.

In expanded form, device ends 115 and 120 may have the form of hollow tubes, for example, to allow blood to flow through the inferior and superior vena cavae after device 100 has been positioned to treat the defect in the septum. In the example illustrated in FIGS. 1A and 1B, device 100 includes two expandable members 130 and 135 that fold out toward the interatrial septum when the central portion 125 of device 100 is expanded. This may increase the area of the interatrial septum that may be covered by the central portion 125 of the device and may also reduce interference with blood flow through the right atrium after device 100 has been deployed. It is not necessary that device 100 include such expandable members, however. Examples of other suitable expanded configurations of device 100 are described later in this section.

In the illustrated example, device 100 may be compressed into a cylindrical shape (FIG. 1A) to fit in an intravascular delivery device such as a conventional catheter suitable for intravascular delivery of devices to the right atrium. Upon delivery to the right atrium, device 100 may self-expand and/or be expanded (e.g., by balloon or pressurized saline, for example) into the configuration shown in FIG. 1B. Where intended for intravascular delivery, in compressed form device 100 may have a diameter of, for example, about 20 mm to about 40 mm (e.g., about 25 mm to about 30 mm) and a length of, for example, about 7 cm to about 15 (e.g., about 10 cm) to allow delivery by conventional intravascular delivery devices. Variations of device 100 may have a range of different expanded sizes/dimensions so that a device having a suitable expanded size for treating a particular patient may be selected. In particular, the expanded dimensions of a device 100 may be chosen so that device ends 115 and 120 may be securely positioned in a particular subject's inferior and superior vena cavae and at least a part of central portion 125 at least partially covers the defect in the subject's interatrial septum.

One of ordinary skill in the art having the benefit of this disclosure could use conventional methods and materials used in the construction of intravascular stents and patches and covered stent grafts, for example, to construct device 100 and variations thereof.

In some variations, device 100 comprises a framework 140 (indicated schematically by cross-hatching in FIGS. 1A, 1B, and subsequent figures) to which other materials (not shown) may be optionally attached. It should be noted that the cross-hatching schematically indicating framework 140 in the various figures is not necessarily intended to indicate the placement of particular framework elements. Framework 140 may be an expandable mesh, for example. Framework 140 may comprise, for example, conventional shape memory materials including, but not limited to, nitinol and stainless steel 316L. Such shape memory materials may allow device 100 to convert between compressed and expanded configurations. In some variations, some or all of framework 140 is bioabsorbable. For example, framework 140 may comprise polylactic-co-glycolic acids and/or other poly orthoesters. Framework 140 may also comprise bioabsorbable metal alloys including, but not limited to, bioabsorbable magnesium metal alloys available from BIOTRONIK, GmbH & Co. K G such as those disclosed, for example, in U.S. patent application Ser. No. 11/221,344, titled “Endoprosthesis Comprising a Magnesium Alloy,” filed Sep. 7, 2005, incorporated herein by reference in its entirety.

Some or all of framework 140 may be formed with wide spacing between framework elements (e.g., a large mesh size) in some variations to allow blood to flow easily through interstices in the framework. In such “wide spacing” variations, the spacing between framework elements may be, for example, greater than about 1 cm, although other larger or smaller spacing may also be used. In one variation, portions of framework 140 not covering or proximate the defect are formed with such wide spacing between framework elements. In some variations, some or all of framework 140 comprises a fine mesh that will block passage of clots, embolic material, or similar debris from or through a defect in the interatrial septum but will allow blood to pass if the mesh were to partly or entirely overlie, for example, the coronary sinus or a valve. In such “fine mesh” variations, the spacing between framework elements may be, for example, less than about 2 mm (e.g., less than about 1 mm or about 1 mm to about 2 mm), although other larger or smaller spacing may also be used. In one variation, some or all of framework 140 in the central portion 125 of device 100 is formed from such a fine mesh. In some variations, some portions of framework 140 are formed with such wide spacing between framework elements and other portions are formed from such a fine mesh.

Device 100 may comprise one or more materials that promote tissue ingrowth into device 100 and/or are conformable to irregularities in atrial surfaces to promote formation of a seal between device 100 and those surfaces. Such materials may be conformable to the surfaces they contact by virtue of being soft, spongy, and or resilient, for example. These materials may also be bioabsorbable. Such conformable materials may include, but are not limited to, Dacron® velour, polyurethane foam, and Vicrylg mesh. Materials that may promote tissue ingrowth may include, but are not limited to, mesh material such as Vicryl®, Dacron® with appropriate pore sizes, and polyvinyl alcohol (PVA) foam meshes. Bioabsorbable materials that may promote tissue ingrowth may include, but are not limited to, bioengineered collagen, caprolactone-co-L-lactide sponge reinforced with knitted poly-L-lactide fabric, and combinations of gelatin and polyglycolic acid (PGA). An example bioengineered collagen is described in “A New Biological Matrix for Septal Occlusion,” Christian Jux et al., Journal of Interventional Cardiology, volume 16, No. 2, 149-152 (2003) incorporated herein by reference in its entirety. Use of a caprolactone-co-L-lactide sponge reinforced with knitted poly-L-lactide fabric and use of combinations of gelatin and polyglycolic acid are described in “Optimal Biomaterial for Creation of Autologuous Cardiac Grafts,” Tsukasa Ozawa et al., Circulation, volume 106, I-176-I-182 (2002), incorporated herein by reference in its entirety.

In some variations, some or all of portions of device 100 that make contact with the interatrial septum (e.g., central portion 125), the inferior or superior vena cavae (e.g., device ends 115, 120), and/or other atrial surfaces include materials that are conformable to the surface they contact and/or promote tissue ingrowth into the contacting portions of device 100. Portions of device 100 comprising such materials and proximate to, or in contact with, atrial or septal surfaces near the defect may become incorporated over time into those surfaces and thus help seal the defect. Portions of device 100 comprising such materials and proximate to or in contact with the walls of the inferior or superior vena cavae may become incorporated into those walls and thus help secure device 100 in position.

In some variations, device 100 comprises a framework 140 that is partially or entirely covered or coated with materials that are conformable to atrial surfaces they contact and/or promote tissue ingrowth. One or more layers of such materials may be attached to the framework, for example. In some variations, device 100 comprises a framework 140 that is partially or entirely covered with Dacron® or a similar material, with a more conforming material on portions of device 100 that oppose the interatrial septum. Some other particular examples of implantable devices comprising such materials are described in additional detail later in this section.

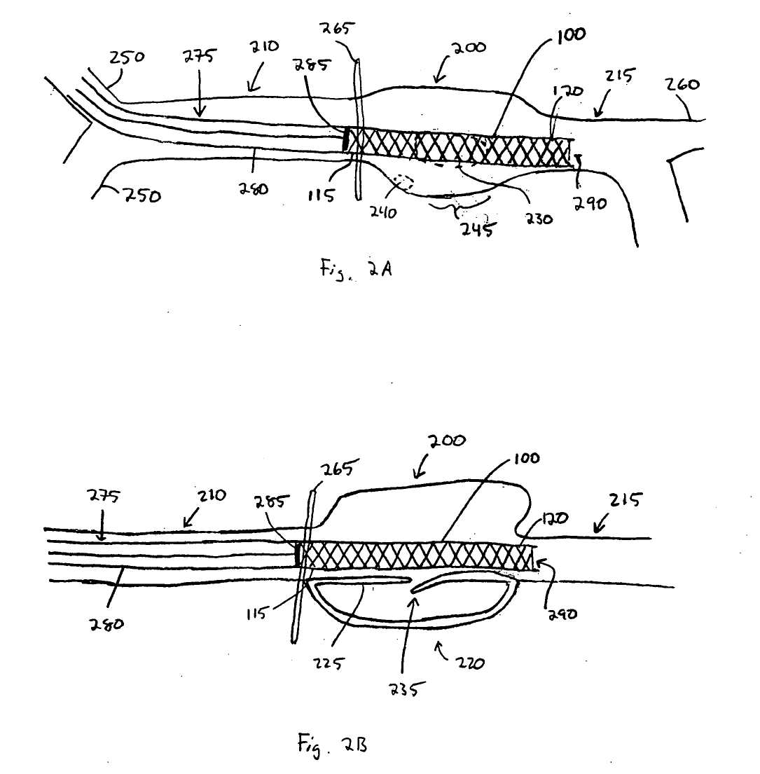

An exemplary method of treating a patent foramen ovale defect in an interatrial septum with a variation of device 100 is now described with reference to FIGS. 2A-6B, in which the “A” and “B” figures show, respectively, top and side views of a subject's right atrium 200, inferior vena cava 210, and superior vena cava 215. These figures also show the left atrium 220, the interatrial septum 225, the fossa ovalis 230, the patent foramen ovale 235, the coronary sinus 240, the area of the tricuspid valve 245, the femoral veins 250, the right internal jugular vein 260, and the diaphragm 265.

For clarity of illustration, only the framework of device 100 is schematically depicted in FIGS. 2A-6B. One of ordinary skill in the art having the benefit of this disclosure will understand that devices incorporating additional materials such as materials that are conformable to atrial surfaces or promote tissue ingrowth as described above, for example, may be used in variations of the exemplary method. The particular examples of such devices described later in this section may be used in variations of the exemplary method.

As a first step, the locations of some or all of the anatomical landmarks shown in FIGS. 2A-6B are obtained using conventional techniques such as, for example, transesophageal echocardiography, intravascular ultrasound (IVUS), and contrast injection during fluoroscopy.

Next, device 100 is compressed and inserted into an intravascular delivery device 275 (see FIG. 2A, for example). Intravascular delivery device 275 comprises a sheath 280 and a plunger 285 that may be used to deploy device 100 through end 290 of sheath 280. Intravascular delivery device 275 may be, for example, a conventional hollow delivery catheter having an inner diameter chosen to accommodate device 100, or a modification thereof. One of ordinary skill in the art having the benefit of this disclosure will be able to choose and/or modify such a conventional delivery catheter as necessary for use with device 100 without undue experimentation. Examples of such conventional delivery systems may include, but are not limited to, those disclosed in U.S. Pat. No. 6,139,572, titled “Delivery System for Intraluminal Vascular Grafts,” issued Oct. 31, 2000, U.S. Pat. No. 6,352,561, titled “Implant Deployment Apparatus,” issued Mar. 5, 2002, and U.S. Pat. No. 6,613,072, titled “Procedures for Introducing Stents and Stent-Grafts,” issued Sep. 2, 2003, each of which is incorporated herein by reference in its entirety.

Referring now to FIGS. 2A and 2B, in the illustrated example delivery device 275 including device 100 is advanced through a femoral vein 250, for example, into the right atrium 200. Delivery device 275 is then positioned in the right atrium such that the portion of delivery device 275 containing end 120 of device 100 is located in the superior vena cava 215 and the portion of delivery device 275 containing end 115 of device 100 is located in the inferior vena cava 210. In some variations, this positioning of delivery device 275 in right atrium 200 is facilitated by observing conventional radio-opaque markings (not shown) on delivery device 275 that indicate the portions of delivery device 275 intended to be positioned in the superior and inferior vena cavae. Such markings may be used to confirm that device 100 is of appropriate size and is correctly positioned before deployment.

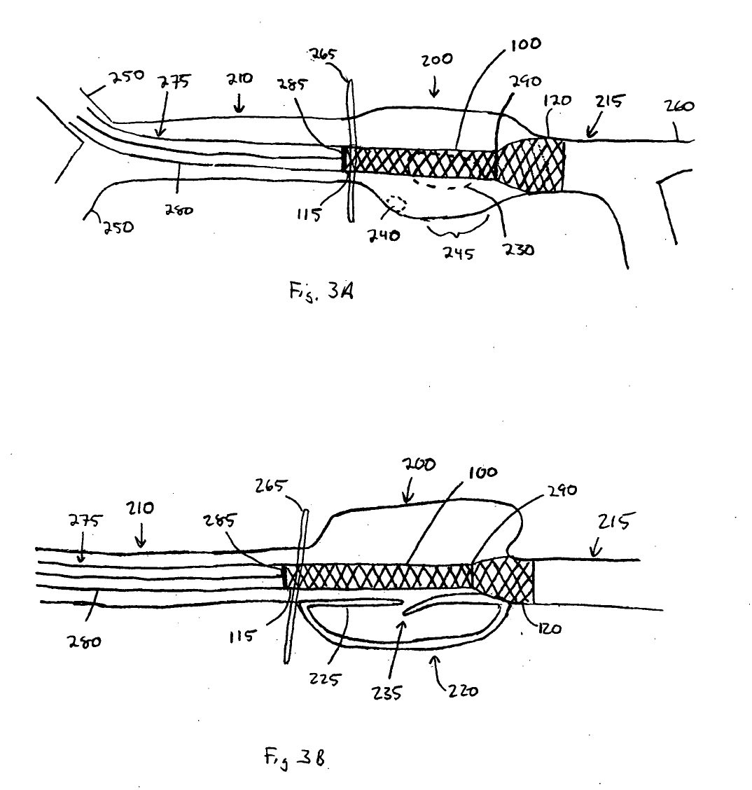

Next, as shown in FIGS. 3A and 3B, the position of device 100 is maintained by plunger 285 while sheath 280 is withdrawn to deploy device 100 from delivery device 275. As sheath 280 is withdrawn, exposed portions of device 100 begin to expand from their compressed to their expanded configurations. FIGS. 3A and 3B show end 120 of device 100 expanding into superior vena cava 215, where radial expansive forces applied by end 120 against the inner wall of superior vena cava 215 secure end 120 in position.

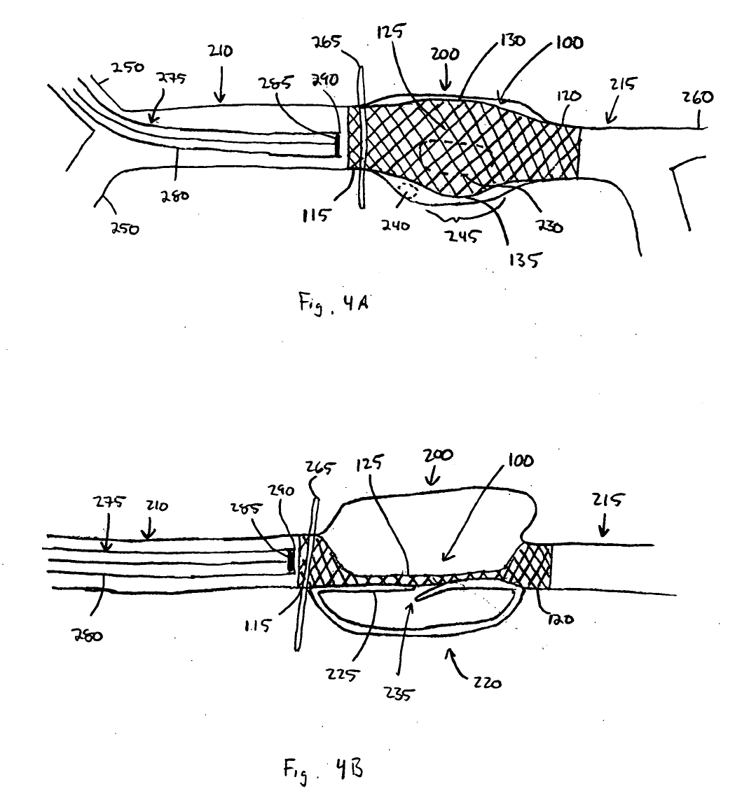

As shown in FIGS. 4A and 4B, as sheath 280 is withdrawn further, central portion 125 of device 100 including expandable members 130 and 135 expands and unfolds to cover patent foramen ovale 235. End 115 of device 100 then expands into inferior vena cava 210 where radial expansive forces applied by end 115 against the inner wall of inferior vena cava 210 secure end 115 in position. In the illustrated example, parts of central portion 125 of device 100 entirely cover fossa ovalis 230, and cover substantially all of the right atrium side of interatrial septum 225, without blocking coronary sinus 240 or the area of the tricuspid valve 245.

After or during deployment of device 100, intravascular ultrasound (IVUS), fluoroscopy, transesophageal echocardiography, or other conventional visualization methods may be used to check the position of device 100 and the apposition of central portion 125 with interatrial septum 225, fossa ovalis 230, and patent foramen ovale 235. A conventional IVUS device (not shown) may be placed in the right atrium 200 via the contralateral femoral vein 250 or via right internal jugular vein 260, for example, by conventional methods. In some variations, if the deployment of device 100 is unsatisfactory, a suture or a stiff member such as a rod, for example, (neither shown) attached to device 100 and attached to or passing through delivery device 275 may be used to reposition device 100 or to withdraw device 100 into delivery device 275. Delivery device 275 may be removed from right atrium 200 and femoral vein 250 after satisfactory deployment of device 100 has been confirmed.

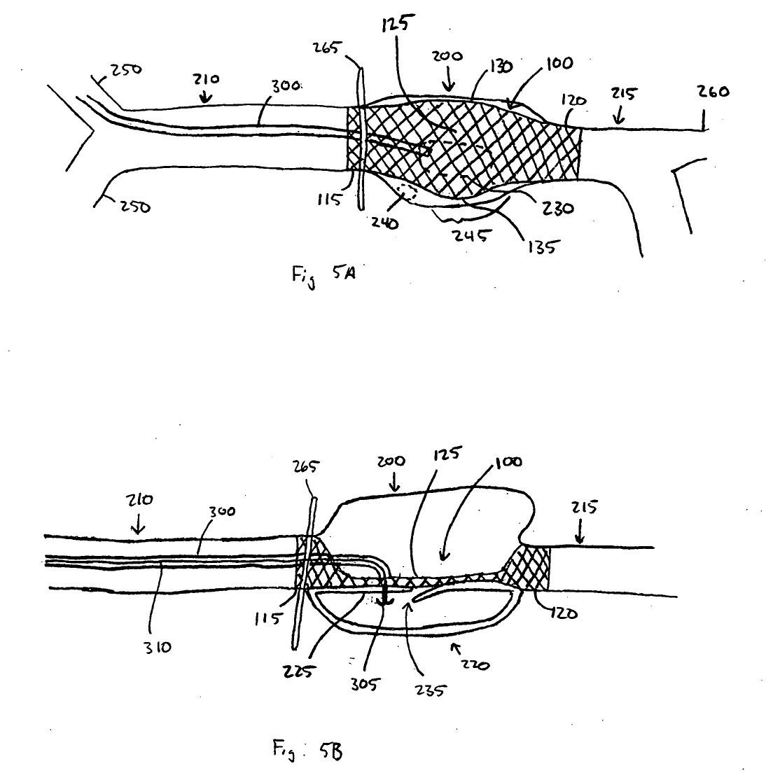

Next, referring to FIGS. 5A and 5B, an intravascular delivery device 300 including at least one anchor, 305 is advanced through a femoral vein 250 into the right atrium, and one or more anchors 305 are placed through central portion 125 of device 100 into interatrial septum 225 or other atrial tissue to secure at least a part of central portion 125 of device 100 to interatrial septum 225 or other atrial tissue. For example, one or more anchors 305 may be placed around the perimeter of central portion 125 of device 100 into interatrial septum 225 and/or through central portion 125 of device 100 into interatrial septum 225 proximate to patent foramen ovale 235. Typically, the choice of positions at which to place anchors 305 is not critical. The placement of the anchors can be checked using conventional visualization methods as described above such as, for example, a conventional IVUS device placed in the right atrium.

In some variations, anchors 305 may be placed anywhere through the central portion 125 of device 100. This may allow tailoring placement of anchors 305 and/or central portion 125 of device 100 to a subject's anatomy without requiring detailed imaging or anatomical information.

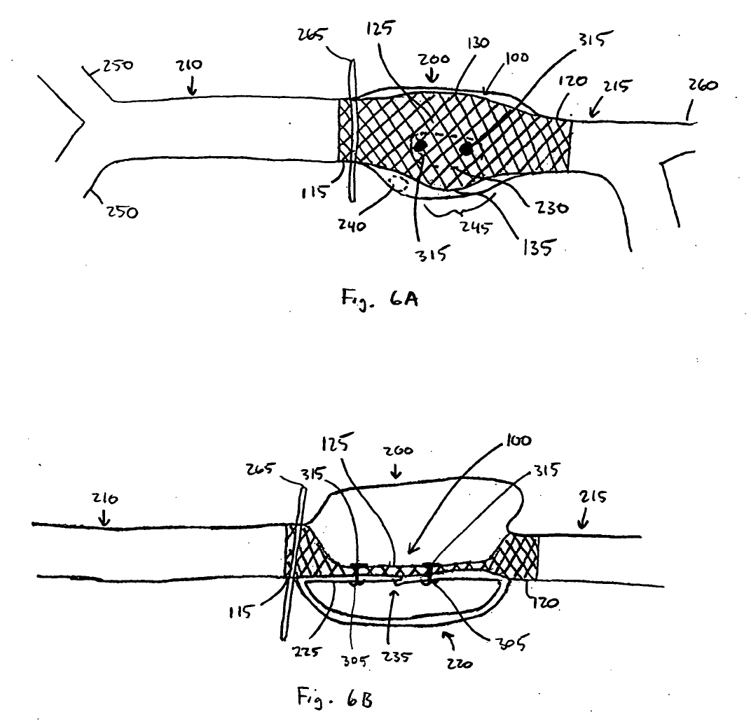

In the illustrated example, anchor 305 is attached to a suture 310, one end of which is retained outside of the subject's body. Tension may be applied to suture 310 to draw anchor 305 and interatrial septum 225 against central portion 125 of device 100 while a “cap” or securing device 315 (FIGS. 6A and 6B) is advanced over suture 310 and deployed against central portion 125 of device 100 to secure anchor 305 in position. A conventional suture cutting device included in or delivered by delivery device 300, for example, may then be used to cut the suture from the anchor. FIGS. 6A and 6B show two anchors 305, secured in position by caps 315, securing central portion 125 of device 100 to interatrial septum 225. More or fewer anchors may be used in other variations.

If, for example, a new leak in patent foramen ovale 235 is detected subsequent to treatment, in some variations an intravascular delivery device 300 may be reintroduced into right atrium 200 to place additional anchors 305 through central portion 125 of device 100 into interatrial septum 225. These additional anchors may further secure central portion 125 of device 100 to interatrial septum 225 and, for example, further seal patent foramen 235.

Anchors 305 and securing devices 315 may be conventional devices used for intravascularly anchoring implantable devices to tissue, for example. Intravascular delivery device 300 may be a conventional delivery catheter suitable for delivering and placing anchors and anchor securing devices, for example. One of ordinary skill in the art having the benefit of this disclosure will be able to choose and/or modify such conventional anchors, anchor securing devices, and conventional delivery catheters as necessary for use anchoring device 100 to interatrial septum 225 without undue experimentation. Examples of such conventional anchors, securing devices, and delivery catheters may include, but are not limited to, those disclosed in U.S. Pat. No. 5,626,614, titled “T-Anchor Suturing Device and Method for Using Same,” issued May 6, 1997, U.S. Pat. No. 5,669,917, titled “Surgical Crimping Device and Method of Use,” issued Sep. 23, 1997, U.S. Pat. No. 6,146,387, titled “Cannulated Tissue Anchor System,” issued Nov. 14, 2000, U.S. Pat. No. 6,997,931, titled “System for Endoscopic Suturing,” issued Feb. 14, 2006, U.S. patent application Ser. No. 10/901,444, titled “Delivery Device and Methods for Heart Valve Repair,” filed Jul. 27, 2004, U.S. patent application Ser. No. 10/955,244, titled “Interlocking Tissue Anchor Apparatus and Methods, filed Sep. 30, 2004, and U.S. patent application Ser. No. 10/958,100, titled “Methods and Devices for Soft Tissue Securement,” filed Oct. 4, 2004, each of which is incorporated herein by reference in its entirety.

Referring again to FIGS. 6A and 6B, in the illustrated example central portion 125 of device 100 covers patent foramen ovale 235 and makes contact with portions of interatrial septum 225 surrounding patent foramen ovale 235. In some variations the parts of device 100 covering patent foramen ovale 235 and contacting surrounding portions of interatrial septum 235 are impermeable to blood flow and consequently seal the patent foramen ovale. In other variations, the parts of device 100 covering patent foramen ovale 235 and contacting surrounding portions of interatrial septum 235 comprise a fine mesh that allows blood to pass but prevents the passage of clots, embolic material, or other debris through or from patent foramen ovale 235 into right atrium 200.

The illustrated example also shows patent foramen ovale 235 closed by two anchors 305 that secure portions of interatrial septum 225 forming foramen ovale 235 against central portion 125 of device 100. This may also seal patent foramen ovale 235. It is not necessary that the defect be closed in this manner, however.

Although FIGS. 6A and 6B show central portion 125 of device 100 making contact with and secured to interatrial septum 225, in some variations this need not occur. In some variations, central portion 125 of device 100 may prevent the passage of clots, embolic material, or other debris from or through patent foramen ovale 235 into right atrium 200 by covering patent foramen ovale 235 even without making contact with patent foramen ovale 235 and/or interatrial septum 225. This can be accomplished, for example, by positioning central portion 125 of device 100 sufficiently close to interatrial septum 225 and covering a sufficiently large area of interatrial septum 225 surrounding patent foramen ovale 235.

The illustrated example shows central portion 125 of device 100 secured to interatrial septum 225 with anchors. In other variations, however, central portion 125 may be secured to interatrial septum 225 or other atrial tissue by other means including, but not limited to, surgical adhesives and sutures. Also, in some variations device 100 is not secured to interatrial septum 225. The secure placement of ends 115 and 120 of device 100 in the inferior and superior vena cavae, respectively, may be sufficient in some variations to maintain central portion 125 of device 100 in position to treat a defect in interatrial septum 225. Radial expansive forces in central portion 125 may be sufficient to maintain the position of central portion 125 with respect to interatrial septum 225. In addition, tissue ingrowth may ultimately secure, or further secure, central portion 125 to interatrial septum 225.

Although in the exemplary method just described device 100 is self-expanding upon deployment, in other variations device 100 may be expanded into its expanded configuration by other means such as, for example, a balloon catheter.

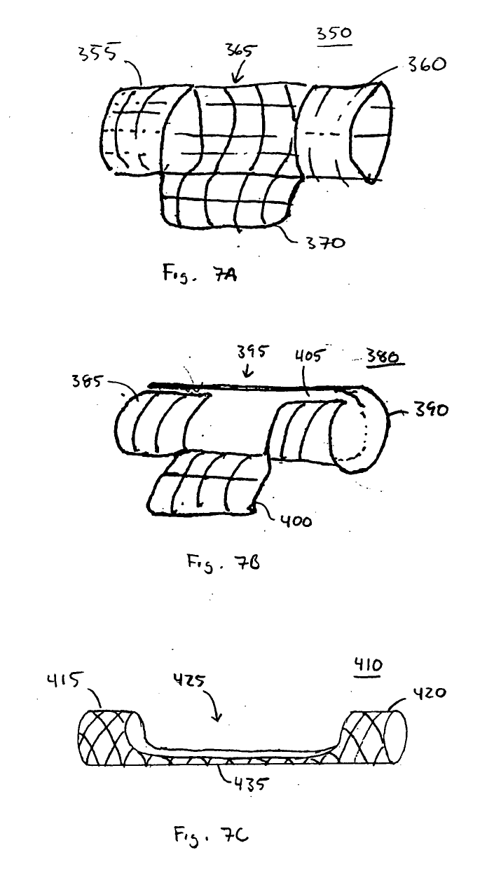

FIGS. 1B and 4A-6B show two similar variations of expanded configurations for implantable device 100. Examples of some other suitable expanded configurations for variations of device 100 are shown in FIGS. 7A-7C. The devices shown in these figures may have compressed forms similar to that shown for device 100 in FIG. 1A, for example.

Device 350, shown in FIG. 7A, comprises ends 355 and 360 that may be expanded to fit securely into the inferior and superior vena cavae, and a central portion 365 including a single expandable member 370 that expands and folds out toward the interatrial septum when central portion 365 is expanded. This may increase the area of the interatrial septum covered by the central portion 365 of the device and may also reduce interference with blood flow through the right atrium after device 350 is deployed. Use of a single expandable member 370 may be mechanically simpler than use of multiple expandable members (as in FIG. 1B, for example). Device 380, shown in FIG. 7B, similarly comprises ends 385 and 390 that may be expanded to fit securely into the inferior and superior vena cavae, and a central portion 395 including an expandable member 400 that expands and folds out toward the interatrial septum when central portion 365 is expanded. In addition, device 380 comprises a slot 405 parallel to its long axis. Slot 405 allows device 380 to expand radially to a broader range of caval sizes.

Device 410, shown in FIG. 7C, comprises ends 415 and 420 expandable to fit securely into the inferior and superior vena cavae, and a central portion 425 comprising a section 435 that expands toward the interatrial septum when central portion 425 is expanded. In expanded form, device 410 has the form of a hollow cylinder comprising a large opening in central portion 425 defined by ends 415, 420 and section 435. The large central opening may reduce interference with blood flow through the right atrium after device 410 has been deployed.

Although for clarity of illustration FIGS. 2A-7C showed only the framework of variations of devices 100, 350, 380, and 410, one of ordinary skill in the art having the benefit of this disclosure will understand that variations of these devices may incorporate additional materials. Such materials may include materials that are conformable to atrial surfaces and/or promote tissue ingrowth as described above, for example. These materials may also be bioabsorbable in some variations. Described next are particular examples of such devices.

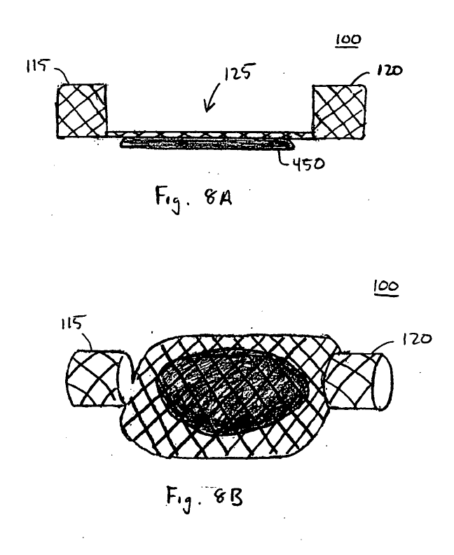

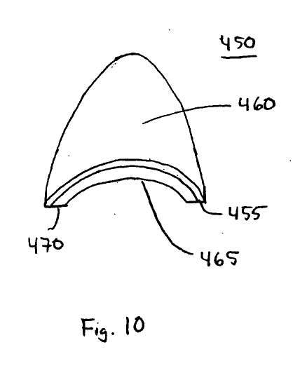

FIGS. 8A and 8B show side and perspective views, respectively, of the expanded configuration of a variation of device 100 comprising a patch 450 attached to the central portion 125 of device 100. Patch 450 may be constructed from one or more compressible materials to allow device 100 including patch 450 to be compressed to fit in an intravascular delivery device. Patch 450 may be impermeable to blood, for example, allowing it to seal a defect in an interatrial septum. As described in greater detail below, patch 450 may have the concave shape shown in the cross-sectional and perspective view of FIG. 10. In some variations, device 100 comprises a framework as described above that is partially or entirely covered with Dacron® or a similar material, and patch 450 is attached to the Dacron® and/or the framework.

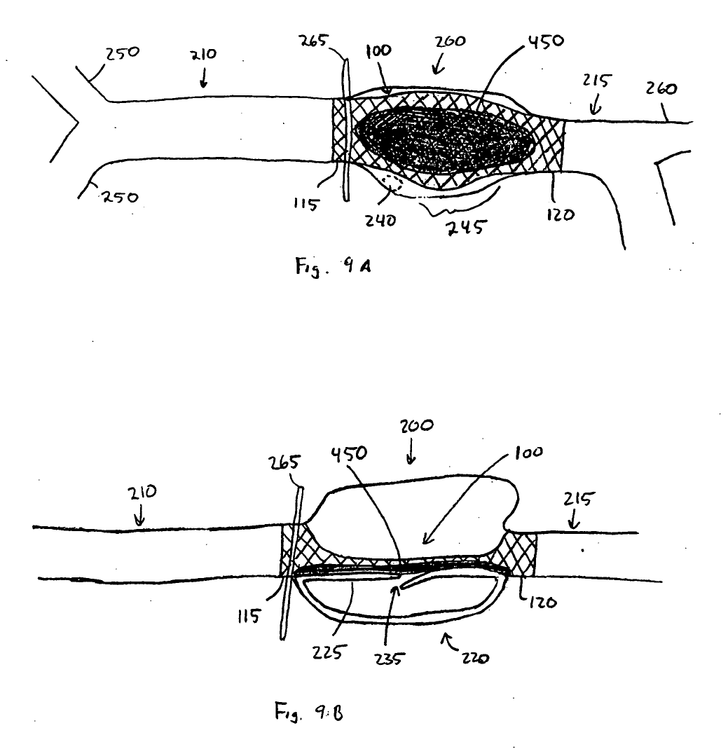

Referring now to FIGS. 9A and 9B, device 100 including patch 450 may be positioned in the right atrium 200 with end 115 in the inferior vena cava 210, end 120 in the superior vena cava 215, and patch 450 covering a defect in the interatrial septum 225 such as, for example, patent foramen ovale 235. In some variations, as shown in FIGS. 9A and 9B, in the expanded configuration central portion 125 pushes patch 450 against interatrial septum 225 to make contact with interatrial septum 225. Such contact may occur, for example, around some or all of the periphery of patch 450, in central portions of patch 450, or both. Patch 450 may be secured to interatrial septum 225 with, for example, anchors, adhesives, or sutures similarly to as described above with respect to other variations of device 100.

The size and shape (e.g., ellipse, circle, rhomboid) of patch 450 may be chosen such that when device 100 including patch 450 is deployed, patch 450 covers substantially all of the interatrial septum 225 without blocking coronary sinus 240 or the area of the tricuspid valve 245. Patch 450 may also cover portions of the walls of inferior vena cava 210 and superior vena cava 220. In other variations, though, patch 450 may cover smaller portions of interatrial septum 225. In some variations, patch 450 may be about 3 cm to about 15 cm long and about 2 cm to about 8 cm wide.

As shown in the cross-sectional and perspective view of FIG. 10, in one variation an elliptical patch 450 comprises a first shaping layer 455 that maintains patch 450 in a convex/concave shape having convex side 460 and concave side 465, and a second layer 470 comprising material that is conformable to irregularities of atrial surfaces and impermeable to blood. Note that the elliptical shape of patch 450 is truncated in FIG. 10 in order to show the cross-section. Convex side 460 of patch 450 may be attached to central portion 125 of device 100 such that concave side 465 faces toward and is pushed against interatrial septum 225 when device 100 is deployed and expanded in right atrium 200. Although the variation of convex/concave patch 450 shown in FIG. 10 is elliptical, in other variations convex/concave patches may be, for example, circular, or rhomboid.

In some variations, the convex/concave shape of the patch is such that downward pressure on one or more areas near the center of the patch will seat the patch against atrial surfaces around the complete perimeter of the patch. Conformable layer 470 may then function as a gasket seal around the edges of patch 450, allowing patch 450 to seal an interatrial defect.

Layer 470 may have a porous structure in some variations to promote tissue ingrowth into patch 450. Patch 450 may also be partially or entirely coated with synthetic or autologous material that promotes tissue ingrowth. In some variations, non-compliant components of patch 450 absorb after sufficient time has passed to allow incorporation of layer 470 into tissue. In other variations, all of patch 450 is made of bioabsorbable material that dissolves as tissue ingrowth occurs until the treated defect is entirely covered with autologuous fibrous tissue.

Shaping layer 455 may comprise, for example, conventional shape memory materials including, but not limited to, nitinol and stainless steel 316L. Shaping layer 455 may comprise a mesh, for example. Layers 455 and 470 may also comprise, for example, the conformable, bioabsorbable, and tissue ingrowth promoting materials listed above in the description of variations of device 100.

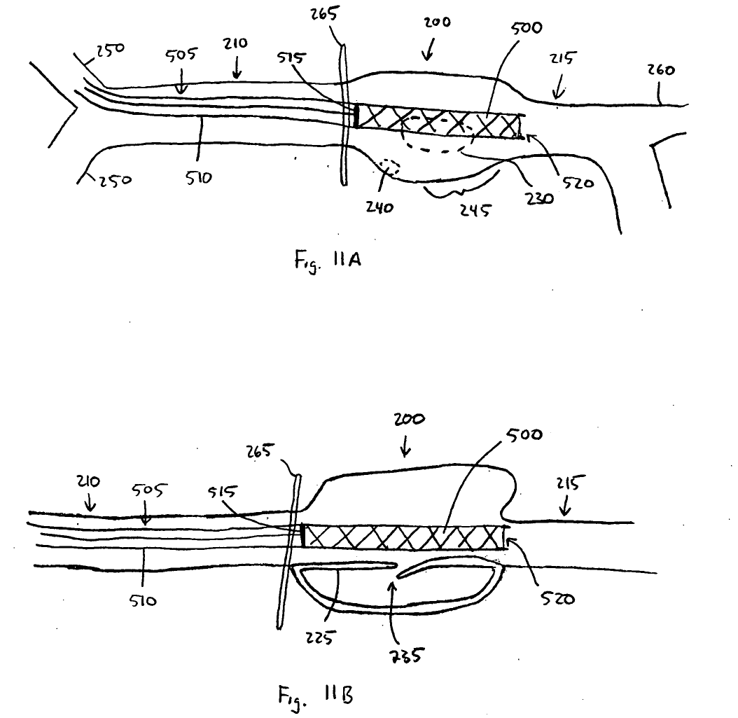

Another exemplary method for treating a patent foramen ovale is described next with reference to FIGS. 11A-14B. In this method a patch such as, for example, patch 450 described above or a variation thereof may be separately deployed (e.g., without other components of device 100) in the right atrium to treat the defect. In some variations, the patch covers substantially all of the right atrium side of the interatrial septum without blocking the coronary sinus or the area of the tricuspid valve. Many of the steps and apparatus of this exemplary method are similar to those of the previously described exemplary method.

As a first step, the locations of some or all of the anatomical landmarks shown in FIGS. 11A-14B are obtained using conventional techniques such as, for example, transesophageal echocardiography, IVUS, and contrast injection during fluoroscopy.

Next, a patch 500 is compressed and inserted into an intravascular delivery device 505 (see FIG. 11A, for example.) Patch 500 may have any of the properties of patch 450 and variations thereof described above, such as a convex/concave shape, for example. Intravascular delivery device 505 comprises a sheath 510 and a plunger 515 that may be used to deploy patch 500 through end 520 of sheath 510. Intravascular delivery device 505 may be, for example, a conventional hollow delivery catheter having an inner diameter chosen to accommodate patch 500, or a modification thereof. One of ordinary skill in the art having the benefit of this disclosure will be able to choose and/or modify such a conventional delivery catheter as necessary for use with patch 500 without undue experimentation. Examples of such conventional delivery systems may include, but are not limited to, those disclosed above with respect to the delivery of device 100.

Referring now to FIGS. 11A and 11B, in the illustrated example delivery device 505 including patch 500 is advanced through a femoral vein 250, for example, into the right atrium 200. Delivery device 505 is then positioned in the right atrium such that the patch, when deployed, will at least partially cover the patent foramen ovale.

In some variations, this positioning of delivery device 505 in right atrium 200 is facilitated by observing conventional radio-opaque markings (not shown) on delivery device 505 that indicate the position of patch 500 in delivery device 505. A comparison of such markings with the positions of anatomical landmarks may be used to confirm that patch 500 is of appropriate size and is correctly positioned before deployment.

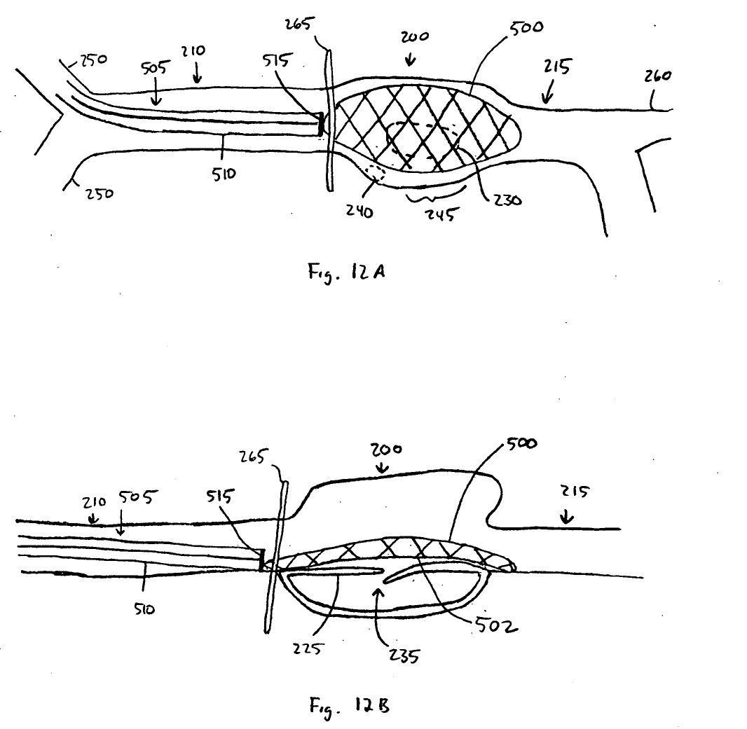

Next, as shown in FIGS. 12A and 12B, the position of patch 500 is maintained by plunger 515 while sheath 510 is withdrawn to deploy patch 500 from delivery device 505. As sheath 515 is withdrawn, patch 500 expands to cover patent foramen ovale 235 and, optionally, surrounding regions of interatrial septum 225. In the illustrated example, patch 500 expands to cover substantially all of interatrial septum 225 as well as portions of the walls of inferior vena cava 210 and superior vena cava 215 with concave side 502 without blocking the coronary sinus 240 or the area of the tricuspid valve 245.

In some variations, patch 500 remains attached to plunger 515 after deployment, allowing manipulation of patch 500 in right atrium 200 by advancing, retracting, or rotating plunger 515, and allowing patch 500 to be held in position during a subsequent step of securing patch 500 to interatrial septum 225. A patch 500 attached to plunger 515 after deployment may also be withdrawn into delivery device 505, in some variations.

After or during deployment of patch 500, intravascular ultrasound (IVUS), fluoroscopy, transesophageal echocardiography, or other conventional visualization methods may be used to check the position of patch 500. A conventional IVUS device (not shown) may be placed in the right atrium 200 via the contralateral femoral vein 250 or via right internal jugular vein 260, for example, by conventional methods. If the deployment of patch 500 is unsatisfactory, the position of patch 500 may be manipulated or patch 500 may be withdrawn as described above.

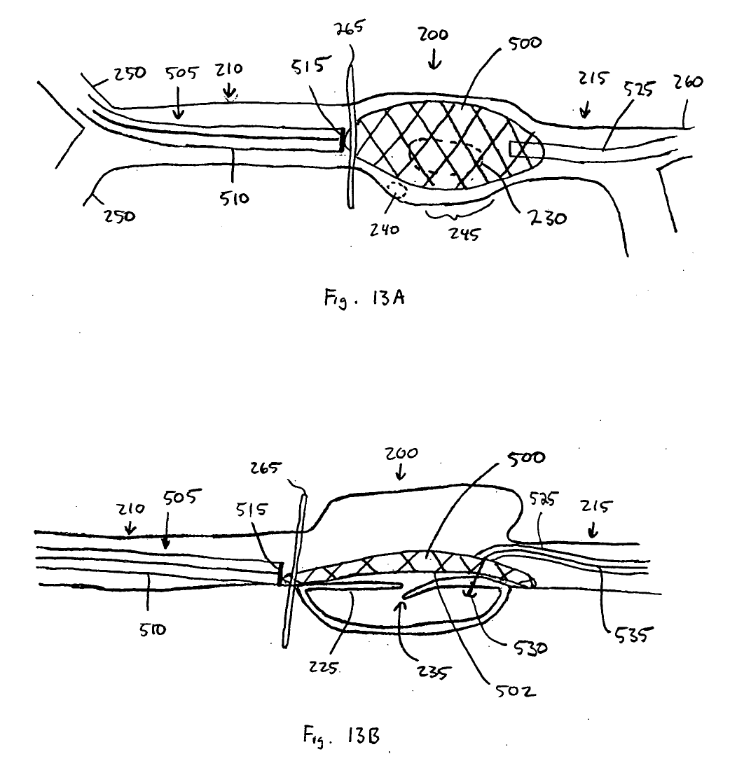

Next, referring to FIGS. 13A and 13B, an intravascular delivery device 525 including at least one anchor 530 is advanced through right internal jugular vein 260 into the right atrium, and one or more anchors 530 are placed through patch 500 into interatrial septum 225 or other atrial tissue to secure at least a part of patch 500 to interatrial septum 225 or other atrial tissue. In some variations, one or more anchors 530 are placed around the perimeter of patch 500 and/or through patch 500 into interatrial septum 225 proximate to patent foramen ovale 235. Typically, the choice of positions at which to place anchors 530 is not critical. The placement of the anchors can be checked using conventional visualization methods as described above such as, for example, a conventional IVUS device placed in the right atrium as described above.

In some variations, anchors 530 may be placed anywhere through patch 500. This may allow tailoring placement of anchors 530 and/or patch 500 to a subject's anatomy without requiring detailed imaging or anatomical information.

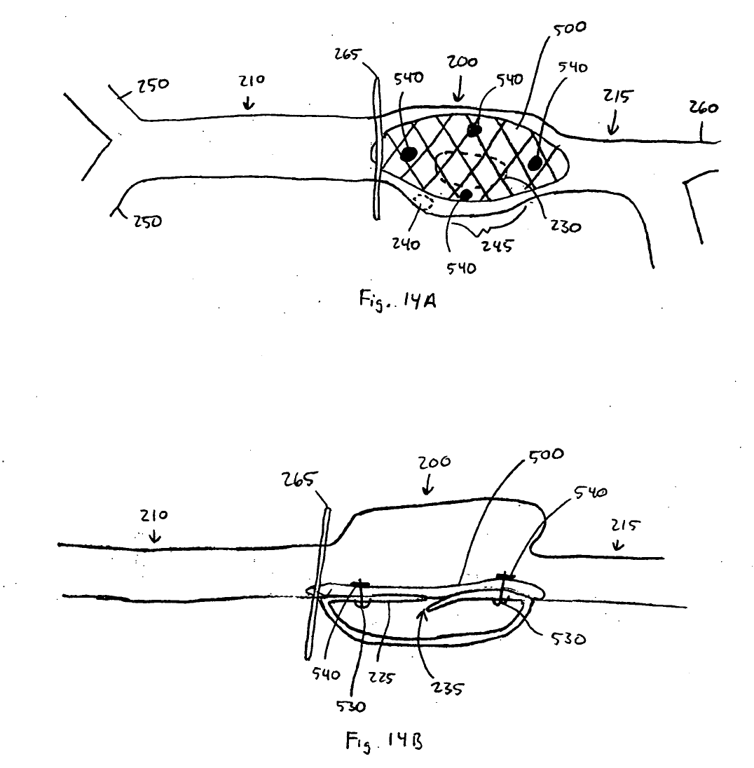

In the illustrated example, anchor 530 is attached to a suture 535, one end of which is retained outside of the subject's body. Tension may be applied to suture 535 while a “cap” or securing device 540 (FIGS. 14A and 14B) is advanced over suture 535 and deployed against patch 500 to press patch 500 against interatrial septum 225 and secure anchor 530 in position. A conventional suture cutting device included in or delivered by delivery device 505, for example, may then be used to cut the suture from the anchor. Anchors 530, securing devices 540, and intravascular delivery device 505 may be conventional devices, or modifications thereof, similar or identical to those used in the previously described exemplary method for treating a patent foramen ovale.

FIGS. 14A and 14B show four anchors 535, secured in position by caps 540, securing patch 500 to interatrial septum 225. More or fewer anchors may be used in other variations. In some variations, only one anchor is used. Once patch 500 is secured in right atrium 200, delivery device 505 may be removed.

If, for example, a new leak in patent foramen ovale 235 is detected subsequent to treatment, in some variations an intravascular delivery device 505 may be reintroduced into right atrium 200 to place additional anchors 530 through patch 500 into interatrial septum 225. These additional anchors may further secure patch 500 to interatrial septum 225 and, for example, further seal patent foramen 235.

Although the illustrated example shows patch 500 secured to interatrial septum 225 with anchors, in other variations patch 500 may be secured to interatrial septum 225 or other atrial tissue by other means including, but not limited to, surgical adhesives and sutures.

In some variations, the methods described herein may be used to stabilize an atrial septal aneurysm. An atrial septal aneurysm is a localized out pouching in the interatrial septum that can occur if the interatrial septum is floppy and/or redundant. Such atrial septal aneurysms may be stabilized, for example, by positioning an implantable patch as described herein to cover the interatrial septum and then placing one or more anchors through the patch into the aneurysmal septum to pull the aneurismal septum up against the patch. This process may be similar to the placement and anchoring of patch 500 shown in FIGS. 14A and 14B, for example. Atrial septal aneurysms may also be stabilized, for example, by anchoring the aneurismal septum to the central portion of an implantable device deployed in the right atrium, inferior vena cava, and superior vena cava. This process may be similar to the placement and anchoring of central portion 125 of device 100 shown in FIGS. 6A and 6B, for example. In some variations, an implantable patch or device may be secured to an interatrial septum to stabilize a septal aneurysm as well as treat another defect such as, for example, a patent foramen ovale.

Variations of the methods described above for treating a defect in an interatrial septum need not include all of the described steps. In some variations, some of the steps described may be executed in parallel or in an order other than that described. Also, in some variations the methods may include additional steps not described in the above examples. Intravascular devices used in the described methods such as delivery devices and visualization devices, for example, may generally be placed in the right atrium via femoral or jugular veins. The particular examples of intravascular access to the right atrium via particular routes should not be taken as limiting.

Intravascular delivery and visualization devices described herein may be introduced into the right atrium, in some variations, by passing them over or through a conventional guide catheter or guide wire previously introduced into the right atrium via the femoral or jugular veins. In some variations, a catheter or guide wire is placed in the coronary sinus to mark the location of the coronary sinus and facilitate placement of a device or patch in the right atrium without covering the coronary sinus.

Although in the exemplary methods described above implantable devices and patches were placed via intravascular routes in the right atrium to treat defects in the interatrial septum, one of ordinary skill in the art having the benefit of this disclosure will understand that the methods and devices described herein may be adapted for use in open heart procedures as well. In such variations, implantable devices and patches as described herein for treating a defect in the interatrial septum need not be compressible to fit into a catheter.

In some variations, a patent ductus arteriosus may be treated with an appropriately sized implantable device 100 and/or patch 450 similar or identical to those described above. Typically, a device or patch used to treat a patent ductus arteriosus will be smaller than a similar device or patch used to treat an atrial defect. One of ordinary skill in the art having the benefit thereof will be able to place the implantable device and/or patch in the proximal descending aorta to treat the patent ductus arteriosus by methods similar to those described above, or variations thereof.

This invention has been described and specific examples of the invention have been portrayed. While the invention has been described in terms of particular variations and illustrative figures, those of ordinary skill in the art will understand that the invention is not limited to the variations or figures described. Therefore, to the extent there are variations of the invention, which are within the spirit of the disclosure or equivalent to the inventions found in the claims, it is the intent that this patent will cover those variations as well. Finally, all publications and patent applications cited in this specification are herein incorporated by reference in their entirety for all purposes as if each individual publication or patent application were specifically and individually put forth herein.

What is claimed is:

1. A method for treating a defect in the septum between the right and left atria of a subject's heart, the method comprising:
providing an implantable device including a first end, a second end, and a central portion; and
positioning the device in the right atrium with the first end in the subject's superior vena cava, the second end in the subject's inferior vena cava, and at least a part of the central portion at least partially covering the defect.

2. The method of claim 1, wherein the defect comprises a patent foramen ovale.

3. The method of claim 1, further comprising contacting the septum with at least a part of the central portion.

4. The method of claim 1, further comprising entirely covering the defect with at least a part of the central portion.

5. The method of claim 1, further comprising sealing the defect with at least a part of the central portion.

6. The method of claim 1, wherein at least a part of the central portion is permeable to blood flow.

7. The method of claim 1, further comprising covering the entire fossa ovalis with at least a part of the central portion without blocking a valve or coronary sinus.

8. The method of claim 7, further comprising covering substantially all of the right atrium side of the septum with at least a part of the central portion without blocking a valve or coronary sinus.

9. The method of claim 1, further comprising securing at least a part of the central portion to the septum at one or more locations proximate to the defect.

10. The method of claim 9, further comprising placing at least one anchor through at least a part of the central portion into the septum.

11. The method of claim 3, further comprising stabilizing a septal aneurysm.

12. The method of claim 1, further comprising introducing the device into the right atrium intravascularly.

13. The method of claim 12, further comprising:
compressing the implantable device to fit into an intravascular delivery device;
introducing the implantable device into the right atrium with the delivery device;
expanding the first end of the implantable device to securely position the first end in the superior vena cava;
expanding the central portion of the implantable device to position at least a part of the central portion to at least partially cover the defect; and
expanding the second end of the implantable device to securely position the second end in the inferior vena cava.

14. The method of claim 13, further comprising intravascularly securing at least a part of the central portion to the septum at one or more locations proximate the defect.

15. The method of claim 14, further comprising delivering the implantable device with a first intravascular delivery device and delivering at least one anchor with a second intravascular delivery device.

16. A device for treating a defect in the septum between the right and left atria of a subject's heart, the device comprising:
a first end expandable to be securely positioned in the subject's inferior vena cava;
a second end expandable to be securely positioned in the subject's superior vena cava; and
a central portion expandable to position at least a part of the central portion to at least partially cover the defect when the first end is securely positioned in the subject's inferior vena cava and the second end is securely positioned in the subject's superior vena cava.

17. The device of claim 16, wherein the defect comprises a patent foramen ovale.

18. The device of claim 16, wherein the device is compressible to fit in an intravascular delivery device.

19. The device of claim 16, wherein at least a part of the central portion is conformable to an atrial surface.

20. The device of claim 16, wherein at least a part of the central portion has a concave shape positionable to face the defect.

21. A method for treating a defect in the septum between the right and left atria of a subject's heart, the method comprising:
providing a patch; and
covering substantially all of the right atrium side of the septum with the patch without blocking a valve or coronary sinus.

22. The method of claim 21, wherein the defect comprises a patent foramen ovale.

23. The method of claim 21, further comprising positioning a concave portion of the patch to face and at least partially cover the defect.

24. The method of claim 21, wherein a septal side of the patch comprises a material conformable to an atrial surface.

25. The method of claim 21, further comprising delivering the patch to the right atrium intravascularly.

26. The method of claim 25, further comprising delivering the patch with a first intravascular delivery device and delivering at least one anchor to anchor the patch to the septum with a second intravascular delivery device.
